(19) United States
(12) Patent Application Publication
(10) Pub. No.: US 2005/0288697 A1
(43) Pub. Date: Dec. 29, 2005

(54) Ring for vitreous surgery for supporting contact lens for the vitreous surgery, cannula used in combination with the ring, and plug used in combination with the ring

(75) Inventors: Mamoru Tei, Kyoto-shi (JP); Hideo Oda, Gamagori-shi (JP)

Correspondence Address: OLIFF & BERRIDGE, PLC, P.O. BOX 19928, ALEXANDRIA, VA, 22320, US

(73) Assignee: NIDEK CO., LTD, Gamagori-shi (JP)

(21) Appl. No.: 11/156,467

(22) Filed: Jun. 21, 2005

(30) Foreign Application Priority Data
Jun. 25, 2004 (JP) 2004-187662

(51) Int. Cl. A61F 9/00

(52) U.S. Cl. 606/166

(57) ABSTRACT

A ring for vitreous surgery, adapted to support a contact lens for the vitreous surgery on a patient's eye, the ring comprises: a ring body; a flange having a contact surface of a curved shape to be fitted with a curved shape of a sclera of the eye, the flange being provided on an outer edge of the ring body; a hole provided in the flange, in which an intraocular insertion surgical instrument is insertable; and a projection provided in the vicinity of the hole on the contact surface of the flange, the projection being able to bite into a conjunctiva of the eye.

### BACKGROUND OF THE INVENTION

1. Field of the Invention

The present invention relates to a surgical instrument for use in vitreous surgery.

2. Description of Related Art

Transconjunctival vitreous surgery has conventionally been performed by use of intraocular insertion surgical instruments such as a 20-gauge vitreous cutter (0.9 mm in diameter). In this surgery, a conjunctiva of a patient's eye is incised and peeled off, and at least three incision wounds for insertion of 20-gauge insertion surgical instruments are made in a sclera of a pars plana of the patient's eye. A vitreous cutter, a light guide, and a cannula are inserted in those incision wounds respectively. The cannula is sutured on the sclera for coming-off prevention thereof. After the surgery, the incision wounds in the sclera are sutured and the conjunctiva is returned to a former state and sutured.

In the surgery, further, a contact lens (CL) for vitreous surgery is supported on the patient's eye by a CL supporting ring, thereby providing a fine view to an operator (surgeon) who conducts the surgery while observing the interior of the patient's eye through the CL and a surgical microscope.

Against the transconjunctival vitreous surgery requiring the suture of the incision wounds, transconjunctival vitreous surgery requiring no suture of the incision wounds by using a 25-gauge insertion surgical instrument (0.5 mm in diameter) to enable the making of a small incision wound has recently been performed. In this surgery, a cylindrical instrument called a trocar is used for facilitating insertion of the insertion instrument, as described in for example a Japanese translation publication No. 2003-526461 of a PCT application (WO 01/068016). The trocar has a double structure that it internally holds a spear-shaped knife. After the conjunctiva is pulled aside, the incision wound is made with the spear-shaped knife. The knife is further stuck into the patients eye, and the trocar is fixed in the incision wound. Even after the knife is removed, the trocar remains held in the incision wound. This makes it possible to ensure an insertion passageway for the insertion instrument. Since the trocar remains held in the incision wound, the position of the incision wound can be clearly found even after removal of the insertion instrument. An outer diameter of the trocar is about 0.65 mm. After the surgery, the conjunctiva having been pulled aside is returned to a former state and the remainder of the vitreous body is incarcerated in the incision wound. Thus, the sutureless surgery can be achieved.

The trocar used in the conventional transconjunctival vitreous surgery is of a thickness of about 0.05 mm to 0.1 mm. If the trocar needs be inserted at the same time of sticking of the spear-shaped knife, the incision wound is forced to largely open by an amount corresponding to the thickness of the trocar. This would cause such problems that insertion of the trocar is difficult for unskilled operators, and the trocar may be deformed, making it hard to provide the insertion passageway for the insertion instrument.

### SUMMARY OF THE INVENTION

The present invention has been made in view of the above circumstances and has an object to overcome the above problems and to provide a ring for vitreous surgery for supporting a contact lens for the vitreous surgery, capable of facilitating recognition of a position of an incision wound and achieving transconjunctival vitreous surgery needing no suture of the incision wound, a cannula and a plug, each being used in combination with the ring.

Additional objects and advantages of the invention will be set forth in part in the description which follows and in part will be obvious from the description, or may be learned by practice of the invention. The objects and advantages of the invention may be realized and attained by means of the instrumentalities and combinations particularly pointed out in the appended claims.

(1) To achieve the purpose of the invention, there is provided a ring for vitreous surgery, adapted to support a contact lens for the vitreous surgery on a patient's eye, the ring comprising: a ring body; a flange having a contact surface of a curved shape to be fitted with a curved shape of a sclera of the eye, the flange being provided on an outer edge of the ring body; a hole provided in the flange, in which an intraocular insertion surgical instrument is insertable; and a projection provided in the vicinity of the hole on the contact surface of the flange, the projection being able to bite into a conjunctiva of the eye.

(2) According to another aspect of the invention, there is provided a cannula which is an intraocular insertion surgical instrument for supplying an infusion liquid into a patient's eye, the cannula being adapted to be used in combination with the above mentioned ring (1), and the cannula comprising a second coming-off prevention device which operates in cooperation with the first coming-off prevention device of the ring.

(3) According to another aspect of the invention, there is provided a plug which is an intraocular insertion surgical instrument for blocking an incision wound which is made in a patient's eye, the plug being adapted to be used in combination with the above mentioned ring (1), and the plug comprising a second coming-off prevention device which operates in cooperation with the first coming-off prevention device of the ring.

(4) Furthermore, according to another aspect, the present invention provides a ring for vitreous surgery, adapted to support a contact lens for the vitreous surgery on a patients eye, the ring comprising: a ring body; a flange having a contact surface of a curved shape to be fitted with a curved shape of a sclera of the eye, the flange being provided on an outer edge of the ring body; a hole provided in the flange, in which an intraocular insertion surgical instrument is insertable; and a first coming-off prevention device for preventing the insertion surgical instrument when inserted in the hole from coming off, the device being provided in at least one of the ring body and the flange.

(5) According to another aspect, the present invention provides a cannula which is an intraocular insertion surgical instrument for supplying an infusion liquid into a patient's eye, the cannula being adapted to be used in combination with the ring (4), and the cannula comprising a second coming-off prevention device which operates in cooperation with the first coming-off prevention device of the ring.

(6) According to another aspect, the present invention provides a plug which is an intraocular insertion surgical instrument for blocking an incision wound which is made in a patient's eye, the plug being adapted to be used in combination with the ring (4), and the plug comprising a second coming-off prevention device which operates in cooperation with the first coming-off prevention device of the ring.

With the ring for supporting the contact lens, the position of the incision wound can be easily recognized, and the transconjunctival vitreous surgery needing no suture of the incision wound can be easily performed. Further, the cannula and the plug make it possible to facilitate the transconjunctival vitreous surgery needing no suture of the incision wound.

### BRIEF DESCRIPTION OF THE DRAWINGS

The accompanying drawings, which are incorporated in and constitute a part of this specification illustrate an embodiment of the invention and, together with the description, serve to explain the objects, advantages and principles of the invention.

In the drawings,

FIG. 1 is a schematic structural view showing the whole of a vitreous surgery system;

FIG. 2 is a perspective external view of a ring for vitreous surgery for supporting a contact lens for the vitreous surgery;

FIG. 3 is a plan view of the ring;

FIG. 4 is a sectional view of the ring taken along a line A-A in FIG. 3;

FIGS. 5A and 5B are explanatory views showing projections formed on a contact surface of a flange;

FIG. 6 is a schematic structural view of a cannula;

FIG. 7 is an explanatory view showing the projections formed on the contact surface of the flange, whereby to prevent movement of a conjunctiva;

FIG. 8 is a schematic structural view of a plug;

FIG. 9 is an explanatory view showing an example of how the plug is used;

FIG. 10A is a view showing another example of the projections formed on the contact surface of the flange; and

FIG. 10B is a sectional view of the contact surface taken in a line B-B in FIG. 10A.

### DETAILED DESCRIPTION OF THE PREFERRED EMBODIMENTS

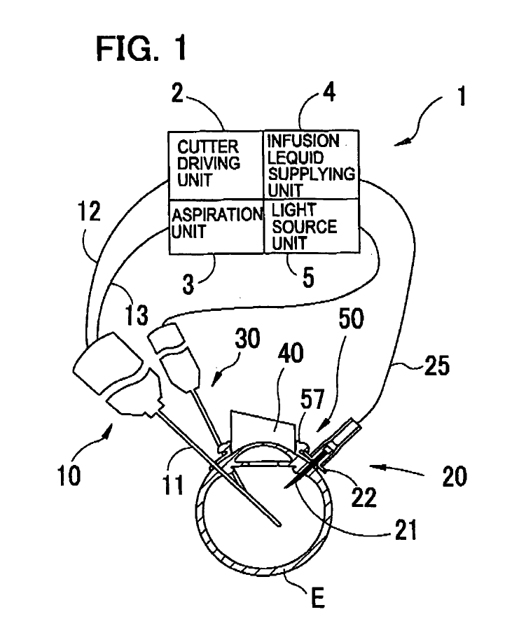

A detailed description of a preferred embodiment of the present invention will now be given referring to the accompanying drawings. FIG. 1 is a schematic structural view showing the whole of a vitreous surgery system.

Connected to a main body 1 of a vitreous surgical apparatus are a vitreous cutter 10, a cannula 20, and a light guide 30, each of which is an intraocular insertion surgical instrument to be inserted in a patient's eye E. The vitreous cutter 10 has a needle 11 having an outer diameter of 23 gauge (0.65 mm) or 25 gauge (0.5 mm) whereby to enable the making of a small incision wound. The needle 11 contains an inner blade movable in an axial direction and also has a distal end with a hole formed on the periphery thereof. The inner blade is moved by air supplied from a cutter driving unit 2 through an air tube 12, thereby exsecting a vitreous body in the eye E. Specifically, the inner blade is internally provided with an aspiration passageway which is communicated with an aspiration unit 3 through an aspiration tube 13. The exsected vitreous body is removed from the eye E through the aspiration tube 13 by operation of the aspiration unit 3. The driving mechanism of the vitreous cutter 10 is well known and therefore its explanation is omitted herein.

The cannula 20 is connected to an infusion liquid supplying unit 4 through an infusion tube 25. This supplying unit 4 is provided in the form of for example a bottle filled with saline and suspended high above the eye E.

The light guide 30 therein includes an optical fiber and is connected to a light source unit 5 to illuminate the interior of the eye E. The outer diameter of a tip end of the light guide 30 which is inserted in the eye E is 23 gauge or 25 gauge (or less) whereby to enable the making of a small incision wound.

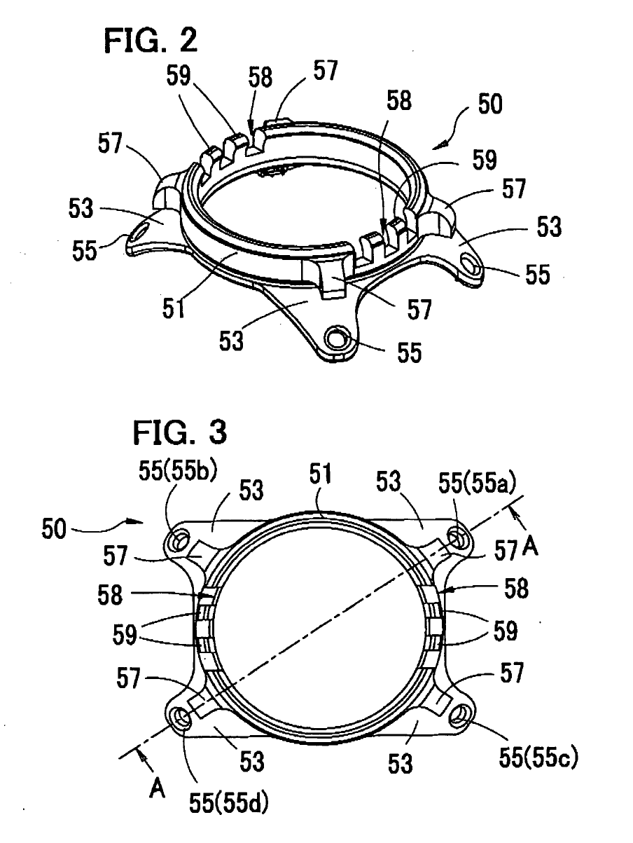

A ring 50 is used to hold or support a contact lens (CL) 40 for vitreous surgery on the eye E. A detailed structure of this ring 50 is explained below with reference to FIGS. 2, 3 and 4. An operator (surgeon) performs the vitreous surgery on the eye E while observing a surgical area of the eye E through the CL 40 and a surgical microscope (not shown).

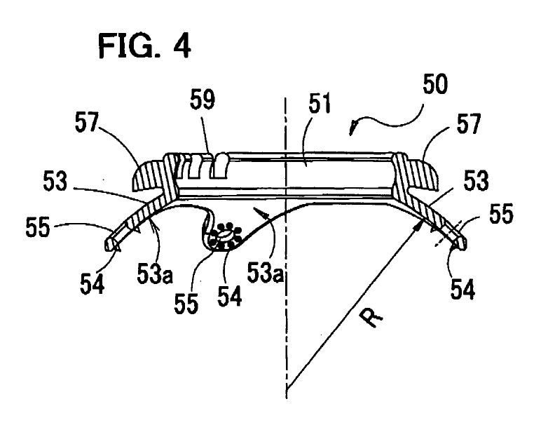

FIG. 2 is a perspective external view of the ring 50. FIG. 3 is a plan view of the ring 50. FIG. 4 is a sectional view of same taken along a line A-A line in FIG. 3. As shown in FIGS. 2, 3, and 4, the ring 50 in the present embodiment includes a ring body 51 having a height of about 2.6 mm and an inner diameter of about 11.2 mm which is slightly larger than the normal diameter of a cornea. The ring body 51 has two sets of three slits 58 cut from above, thus forming two protrusions 59 between the slits 58. The two sets of three slits 58 are arranged in diametrically opposite relation. The protrusions 59 are used as pieces over which a suture thread is hooked in order to suture the ring 50 on the eye E.

The ring body 51 further includes four flanges 53 each extending outward from the bottom edge of the ring body 51. Each flange 53 is formed with four holes (ports) 55 in which the needle 11 of the vitreous cutter 10, a needle 21 of the cannula 20, and others are individually inserted. In the present embodiment, the four holes 55 are two holes 55a and 55b provided on an upper side with respect to the protrusions 519 in FIG. 3 and two holes 55 and 55d provided on a lower side. The holes 55a and 55b are in symmetrical relation, and the holes 55c and 55d are also in symmetrical relation. Further, the holes 55a and 55d are in diagonally opposite relation, and the holes 55b and 55c are also in diagonally opposite relation. Each hole 55 is designed to have a diameter such that the vitreous cutter 10 and the light guide 30, each having 25 gauge or 23 gauge, can be inserted at a desired angle into any hole 55. The diameter of each hole 55 is about 1.0 mm in the present embodiment. Each hole 55 is formed in an appropriate portion of each flange 53 so that each hole 55 is positioned on a pars plana of the eye E when the ring 50 is placed on the eye E. In the present embodiment, specifically, the center of each hole 55 is at a distance of about 3.5 mm from the inner periphery of the ring body 51.

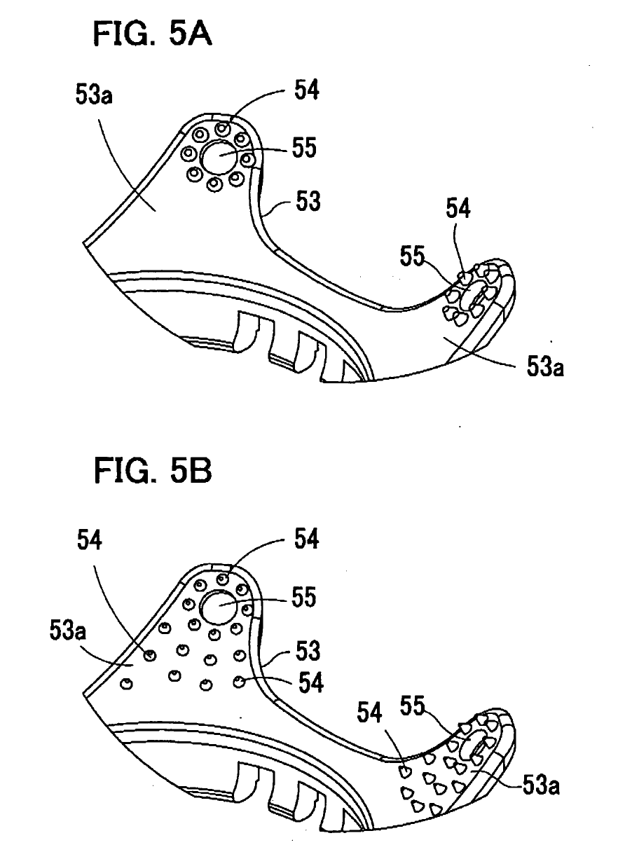

Each flange 53 has a bottom surface serving as a contact surface 53a with respect to the eye E. The contact surface 53a is formed in a curved shape having a predetermined radius of curvature R so that it fits with the curved surface of a sclera of the eye E when the ring 50 is placed thereon. The radius of curvature R of the contact surface 53a is about 12 mm in the present embodiment. Near or around the hole 55 in each contact surface 53a, as shown in FIG. 5A, a plurality of minute projections 54 are provided. Each projection 54 has a slightly sharp-pointed end and, in the present embodiment, has a diameter of about 0.2 to 0.4 mm and a height of about 0.2 to 0.4 mm (substantially equal to the thickness of a conjunctiva).

Formed near each hole 55 is a lug 57 radially extending from the ring body 51. This lug 57 may be provided directly on each flange 53. The lugs 57 constitute a mechanism for preventing the cannula 20 and a plug 70 mentioned later from coming off.

The ring 50 constructed as above is manufactured in one piece by cutting or moulding. The ring 50 is generally made of metal, preferably, of a transparent material such as resin. In this case, at least the flange 53 is entirely or partially formed of the transparent material so that a portion surrounding the hole 55 is transparent. Such entirely or partially transparent flange 53 allows the operator to easily observe the condition of the eye E, e.g., the conjunctiva under the flange 53 and a state of an incision wound to be made through each hole 55.

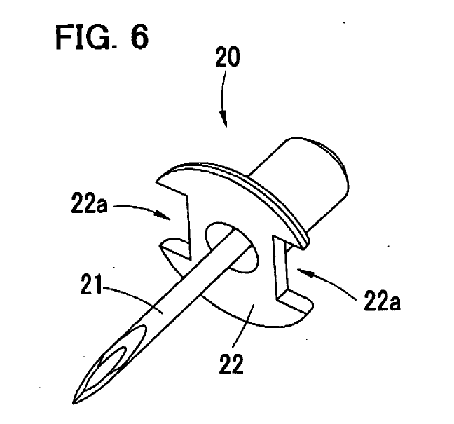

The cannula 20 has a specific structure for use in combination with the above mentioned ring 50. FIG. 6 is a schematic structural view of the cannula 20. In FIG. 6, a flange 22 is formed in a circular shape having two cutouts 22a in diametrically opposite relation. Each cutout 22a has a size enough to allow the lug 57 to pass through when the needle 21 of the cannula 20 is inserted in the hole 55 of the ring 60. To be specific, either of the cutouts 22a of the cannula 20 is aligned with the lug 57 and then the needle 21 is inserted in the hole 55 into the eye E until the flange 22 comes into contact with the flange 53 of the ring 50. After that, the cannula 20 is turned to engage the flange 22 beneath the lug 57. The cannula 20 is thus fixed to the ring 50 and can be prevented from coming off. The lug 57 serves as a fixing member of the coming-off prevention mechanism and the flange 22 serves as an engagement member with respect to the fixing member.

The outer diameter of the needle 21 of the cannula 20 in the present embodiment is 23 gauge or 25 gauge (or less) whereby to enable the making of a small incision wound. The needle 21 has a sharp-pointed end like a syringe needle so that the needle 21 be easily stuck in the sclera through the conjunctiva. In the present embodiment, for example, the needle 21 has a length of about 8 mm from the flange 22 to the sharp-pointed tip and an obliquely cut end of the needle 21 has a length of about 3 mm. The cut end is formed at a sharp angle less than 20°. The cannula 20 with the needle 21 having the sharply-angled end can directly be inserted in the eye E without a cutting instrument such as a spear-shaped knife. This makes it possible to facilitate the surgery. The length of the needle 21 to its tip and the length of the cut end are merely examples and may be shorter than above as long as the needle 21 can directly be inserted in the eye E.

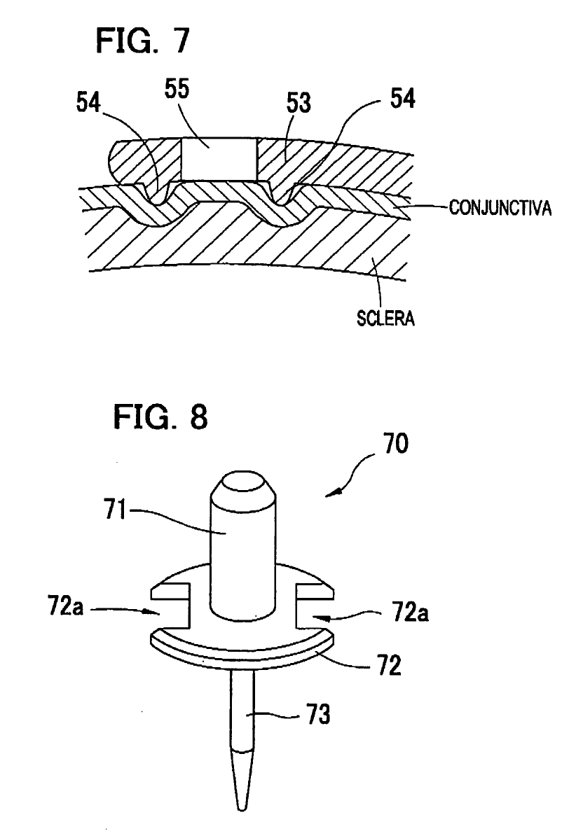

The following explanation is made on the vitreous surgery using the surgical instruments such as the ring 50. The ring 50 placed on the eye E is first fixedly sutured thereon with the suture thread. The position of the ring 50 with respect to the eye E is determined so that the side of the ring 50 having the holes 55a and 55b is positioned on the head side of the patient because the operator usually takes his position on the patient's head side. The holes 55a and 55b are used for insertion of the vitreous cutter 10 and the light guide 30. The holes 55c and 55d are used for insertion of the cannula 20. Specifically, the flange 53 having the hole 55c or 55d is slightly picked up, the conjunctiva under the flange 53 is pulled aside with forceps or the like, and the needle 21 of the cannula 20 is stuck in the eye E through the hole 55c or 55d. The needle 21 having a sharp-pointed tip can directly be stuck in without making an incision wound in advance. Either of the cutouts 22a of the cannula 20 is aligned with the lug 57, and the cannula 20 is stuck and then turned to bring the flange 22 under the lug 57 for engagement therewith. Thus, the cannula 20 can be fixed easily. When the flange 53 picked up is put down onto the eye E, as shown in FIG. 7, the projections 54 formed on the contact surface 53a bite into the conjunctiva, thereby holding down the conjunctiva against the sclera. This makes it possible to fix the conjunctiva under the flange 53 and prevent the movement (return) of the conjunctiva having been pulled aside.

In the other holes 55a and 55b, the vitreous cutter 10 and the light guide 30 are inserted respectively. Specifically, the conjunctiva under the flanges 53 having the holes 55a and 55b are pulled aside with the forceps or the like in the same manner as above, and then the contact surfaces 53a with the projections 54 are put to hold down the conjunctiva having been pulled aside. The conjunctiva can thus be kept in the displaced state. Through each of the holes 55a and 55b, an incision wound is made with the spear-shaped knife, a syringe needle, or the like of 23 or 25 gauge. Subsequently, the vitreous cutter 10 is inserted in one hole 55 (55a) and the light guide 30 is inserted in the other hole 55 (55b). The conjunctiva is held down by each flange 53 at that time as described above, so that the vitreous cutter 10 and the light guide 30 can be inserted easily.

As above, the operator inserts the insertion surgical instruments needed for the surgery in the eye E, places the CL 40 in the ring body 51, operates the vitreous cutter 10 while observing the interior of the eye E through the microscope and the CL 40 to exsect and remove the vitreous body. During the surgery, the vitreous cutter 10 and the light guide 30 may be extracted and inserted for interchange of their inserting positions or for administration by injection into the eye E through the incision wound. The positions of the incision wounds are clarified by the holes 55 of the ring 50, thereby making it easy for the operator to find the position of each incision wound in the extracting and inserting operations.

After the surgery, the inserted instruments (the vitreous cutter 10, the cannula 20, and others) are extracted and the ring 50 is removed. Thus, the conjunctiva having been held in the displaced state by the flanges 53 and the projections 54 is returned to an initial state so that here the conjunctiva covers the incision wounds made in the sclera, resulting in auto-occlusion of the incision wounds. The remainder of the vitreous body is impacted in the incision wound. Consequently, the surgery needing no suture of incised sclera and conjunctiva can be achieved.

FIG. 8 is a schematic structural view of the plug for plugging the incision wound to prevent outflow of intraocular fluid through the incision wound after the inserted light guide 30 or another inserted instrument is extracted therefrom. The plug 70 is used for preventing collapse of the eye E which may be caused by the outflow of the intraocular fluid. The plug 70 is basically constructed of a shaft 71, a flange 72, and a pin 73 which is stuck in the eye E. The pin 73 is of a rod-like shape having a slightly tapered end and a diameter of 23 or 25 gauge. The flange 72 is of the same shape as the flange 22 of the cannula 20. In other words, the flange 72 is provided with two cutouts 72a in diametrically opposite relation. Each cutout 72a is of a size enough to allow each lug 57 of the ring 50 to pass through.

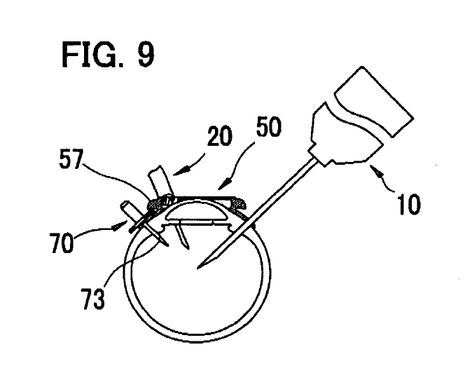

This plug 70 is used as below. For instance, exsection of the vitreous body near a ciliary body is performed by extracting the light guide 30 and instead using epi-illumination of the microscope. At this time, a portion to be exsected is hard to view due to an iris and therefore the surgery is performed by applying external pressure to the eye E with a cotton swab or the like so that the eye E be collapsed inwardly. For this surgery, the pin 73 of the plug 70 is inserted in the incision wound through the hole 55 after extraction of the light guide 30 therefrom, and the cutout 72a is aligned with the lug 57 and the plug 70 is inserted until the flange 72 comes into contact with the flange 53. Then, the plug 70 is entirely turned with forceps or the like to engage the flange 72 beneath the lug 57. The plug 70 is thus fixed to the ring 50 as shown in FIG. 9 and can be prevented from coming off. The inserted pin 73 blocks the incision wound, preventing outflow of the intraocular fluid through the incision wound. The lug 57 serves as a fixing member of the coming-off prevention mechanism and the flange 72 serves as an engagement member with respect to the fixing member.

The present invention may be embodied in other specific forms without departing from the spirit or essential characteristics thereof. For instance, the coming-off prevention mechanisms for the cannula 20 and the plug 70 may be provided as a screw-in mechanism. This may be achieved by forming a female screw thread in each hole 55 and a male screw thread on each outer periphery of the flange 20 of the cannula 20 and the flange 72 of the plug 70 respectively.

The holes 55 serving as insertion ports of the insertion surgical instruments are provided at least three for insertion of the vitreous cutter 10, cannula 20, and light guide 30. The number of holes 55 may be increased as needed for insertion of other insertion instruments or selection of the insertion ports. The vitreous surgery is normally performed by making three incision wounds in the sclera, though the ring 50 is formed with four holes (ports) in the above embodiment. This enables selection of the inserting position of the cannula 20 based on which eye, right or left, is subjected to the surgery.

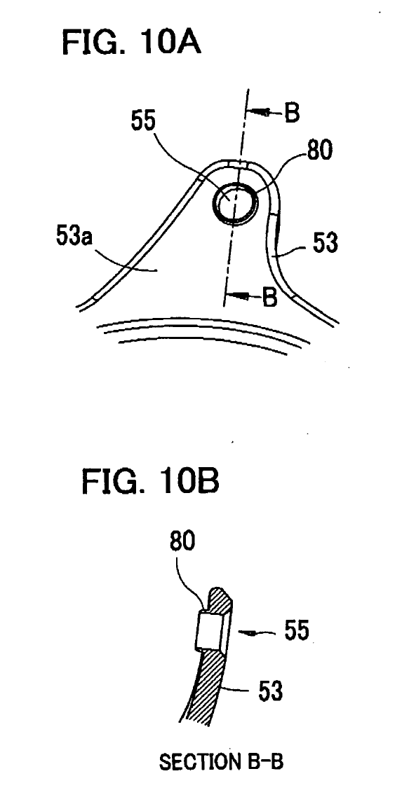

The arrangement of the projections 54 which bite into the conjunctiva pulled aside in order to prevent movement of the conjunctiva is not limited to the above embodiment. The distribution area of projections 54 may be added in other places of the contact surface 53a. For example, besides the surrounding of each hole 55, the projections 54 may be provided widely up to the vicinity of the ring body 51 as shown in FIG. 5B. This ensure the prevention of movement of the conjunctiva. The shape of each projection 54 is also not limited to the illustrated one in FIGS. 5A and 5B. Instead, as shown in FIGS. 10A and 10B, a projection 80 may be provided projecting in a ring shape, slightly tapered externally to an open end, around each hole 55 in the contact surface 53a. This projection 80 has a_thickness of about 0.3 mm and a height of about 0.2 to 0.4 mm (substantially equal to the thickness of the conjunctiva). Further, this ring-shaped projection 80 may be provided in combination with the minute projections 54 which are widely arranged up to the vicinity of the ring body 51 as shown in FIG. 5B.

While the presently preferred embodiment of the present invention has been shown and described, it is to be understood that this disclosure is for the purpose of illustration and that various changes and modifications may be made without departing from the scope of the invention as set forth in the appended claims.

1. A ring for vitreous surgery, adapted to support a contact lens for the vitreous surgery on a patient's eye, the ring comprising:
a ring body;
a flange having a contact surface of a curved shape to be fitted with a curved shape of a sclera of the eye, the flange being provided on an outer edge of the ring body;
a hole provided in the flange, in which an intraocular insertion surgical instrument is insertable; and
a projection provided in the vicinity of the hole on the contact surface of the flange, the projection being able to bite into a conjunctiva of the eye.

2. The ring according to claim 1, wherein
the flange includes a transparent portion surrounding the hole.

3. The ring according to claim 1, further comprising
a first coming-off prevention device for preventing the insertion surgical instrument when inserted in the hole from coming off, the device being provided in at least one of the ring body and the flange.

4. A cannula which is an intraocular insertion surgical instrument for supplying an infusion liquid into a patient's eye, the cannula being adapted to be used in combination with the ring as set forth in claim 3, and the cannula comprising a second coming-off prevention device which operates in cooperation with the first coming-off prevention device of the ring.

5. The cannula according to claim 4, wherein the second coming-off prevention device operates in engagement with the first coming-off prevention device of the ring.

6. The cannula according to claim 4 comprising a needle for injecting the infusion liquid into the eye, the needle having a sharp-pointed end.

7. A plug which is an intraocular insertion surgical instrument for blocking an incision wound which is made in a patient's eye, the plug being adapted to be used in combination with the ring as set forth in claim 3, and the plug comprising a second coming-off prevention device which operates in cooperation with the first coming-off prevention device of the ring.

8. The plug according to claim 7, wherein the second coming-off prevention device operates in engagement with the first coming-off prevention device of the ring.

9. A ring for vitreous surgery, adapted to support a contact lens for the vitreous surgery on a patient's eye, the ring comprising:
a ring body;
a flange having a contact surface of a curved shape to be fitted with a curved shape of a sclera of the eye, the flange being provided on an outer edge of the ring body;
a hole provided in the flange, in which an intraocular insertion surgical instrument is insertable; and
a first coming-off prevention device for preventing the insertion surgical instrument when inserted in the hole from coming off, the device being provided in at least one of the ring body and the flange.

10. The ring according to claim 9, wherein
the flange includes a transparent portion surrounding the hole.

11. A cannula which is an intraocular insertion surgical instrument for supplying an infusion liquid into a patient's eye, the cannula being adapted to be used in combination with the ring as set forth in claim 9, and the cannula comprising a second coming-off prevention device which operates in cooperation with the first coming-off prevention device of the ring.

12. The cannula according to claim 11, wherein the second coming-off prevention device operates in engagement with the first coming-off prevention device of the ring.

13. A plug which is an intraocular insertion surgical instrument for blocking an incision wound which is made in a patient's eye, the plug being adapted to be used in combination with the ring as set forth in claim 9, and the plug comprising a second coming-off prevention device which operates in cooperation with the first coming-off prevention device of the ring.

14. The plug according to claim 13, wherein the second coming-off prevention device operates in engagement with the first coming-off prevention device of the ring.
